(19) United States
(12) Patent Application Publication
(10) Pub. No.: US 2023/0292850 A1
(43) Pub. Date: Sep. 21, 2023

(54) MOISTURIZING FACILITATING FOOT COVERING ASSEMBLY

(71) Applicants: Hayat Ibrahim, Saint Paul, TX (US); Sara Sheikh, Saint Paul, TX (US)

(72) Inventors: Hayat Ibrahim, Saint Paul, TX (US); Sara Sheikh, Saint Paul, TX (US)

(21) Appl. No.: 17/695,327

(22) Filed: Mar. 15, 2022

(51) Int. Cl. A41B 11/00; A61F 13/06; A61F 13/00

(52) U.S. Cl. CPC A41B 11/007; A41B 11/005; A61F 13/064; A61F 13/00017; A61F 13/00029; A61F 13/00046

(57) ABSTRACT

A moisturizing facilitating foot covering assembly includes a sleeve of cloth material to be positioned on and cover a person's foot. The sleeve includes a bottom wall and a perimeter wall that is attached to and extends upwardly from the bottom wall. The perimeter wall includes a heel end, a toe end, a first lateral edge and a second lateral edge. A top wall is attached to the perimeter wall. The top wall has a foot opening therein positioned adjacent to the heel end. The cloth material is air and fluid permeable. A coating is positioned on an interior surface of the sleeve. The coating is flexible as well as air and fluid impermeable. The top wall is free of the coating. The coating prevents the sleeve from absorbing any moisturization composition positioned on a lower surface of the person's foot.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to foot covering device and more particularly pertains to a new foot covering device for retaining moisturizing agents against a person's foot in a comfortable manner.

### (2) Description of Related Art Including Information Disclosed Under 37 CFR 1.97 and 1.98

The prior art relates to foot covering devices used to cover a person's foot after the foot has been treated with a moisturizing agent. These typically include socks having a greater thickness but which do not prevent the sock from absorbing the moisturizing agent. Alternatively, a person may cover their foot with a fluid impermeable plastic bag or plastic sheets which is uncomfortable and results in the foot sweating and inhibiting the moisturizing process.

### BRIEF SUMMARY OF THE INVENTION

An embodiment of the disclosure meets the needs presented above by generally comprising a sleeve of cloth material to be positioned on and cover a person's foot. The sleeve includes a bottom wall and a perimeter wall that is attached to and extends upwardly from the bottom wall. The perimeter wall includes a heel end, a toe end, a first lateral edge and a second lateral edge. A top wall is attached to the perimeter wall. The top wall has a foot opening therein positioned adjacent to the heel end. The cloth material is air and fluid permeable. A coating is positioned on an interior surface of the sleeve. The coating is flexible as well as air and fluid impermeable. The top wall is free of the coating. The coating prevents the sleeve from absorbing any moisturization composition positioned on a lower surface of the person's foot.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a side view of a moisturizing facilitating foot covering assembly according to an embodiment of the disclosure.

FIG. 2 is a top view of an embodiment of the disclosure.

FIG. 3 is a cross-sectional view of an embodiment of the disclosure taken along line 2-2 of FIG. 1.

FIG. 4 is a cross-sectional view of an embodiment of the disclosure taken along line 4-4 of FIG. 2.

FIG. 5 is a side view of a second embodiment of the disclosure.

FIG. 6 is a top view of the second embodiment of the disclosure.

FIG. 7 is a cross-sectional view of the second embodiment of the disclosure taken along line 7-7 of FIG. 5.

FIG. 8 is a cross-sectional view of the second embodiment of the disclosure taken along line 8-8 of FIG. 6.

### DETAILED DESCRIPTION OF THE INVENTION

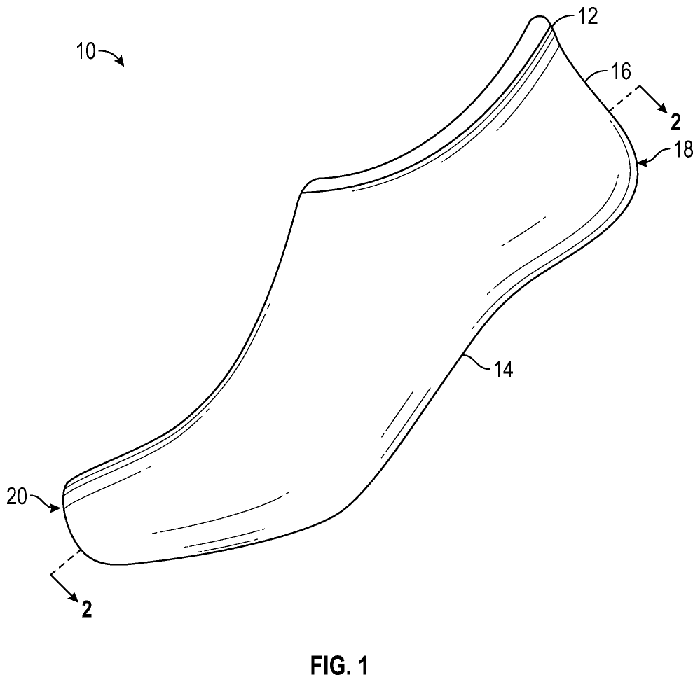

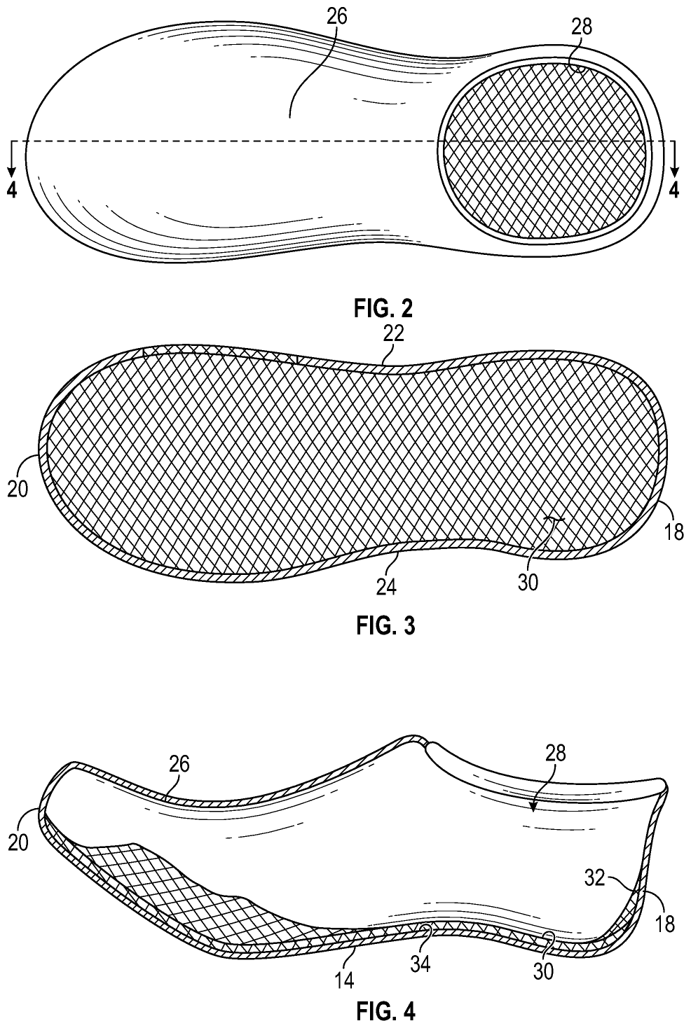

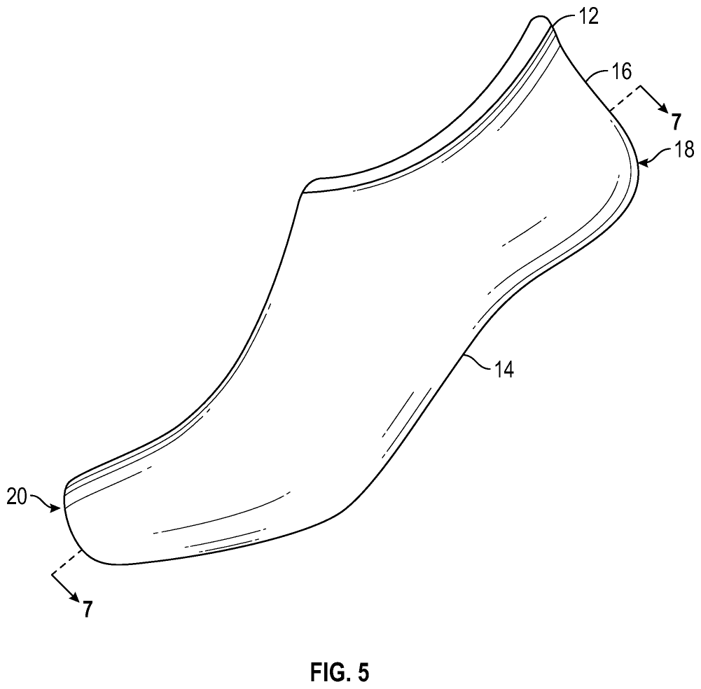

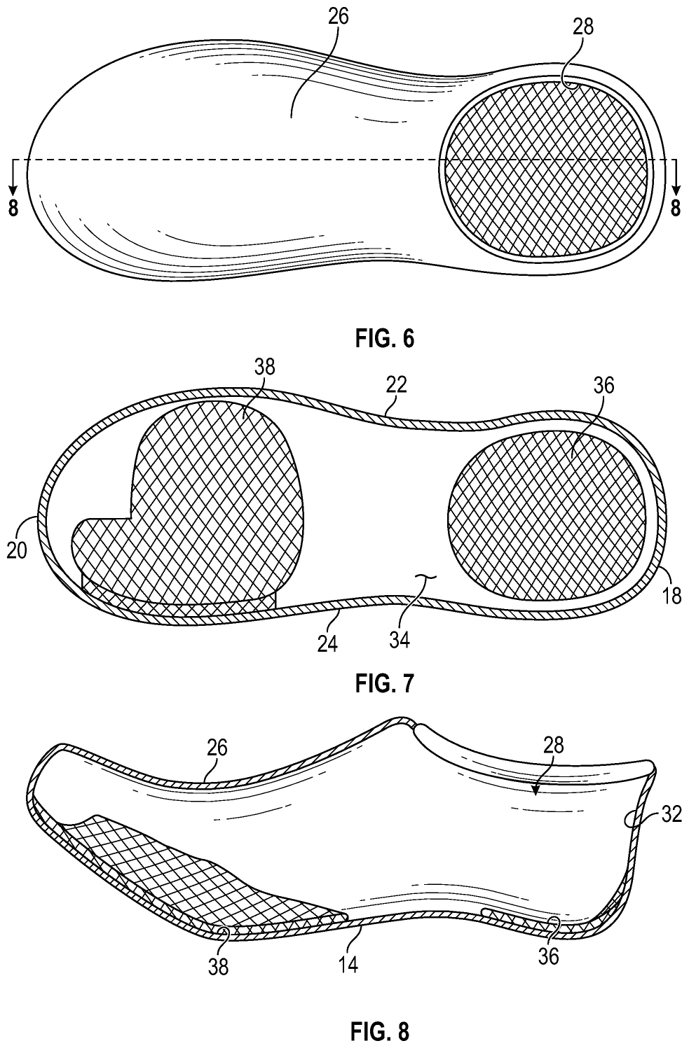

With reference now to the drawings, and in particular to FIGS. 1 through 8 thereof, a new foot covering device embodying the principles and concepts of an embodiment of the disclosure and generally designated by the reference numeral 10 will be described.

As best illustrated in FIGS. 1 through 8, the moisturizing facilitating foot covering assembly 10 generally comprises a sleeve 12 of a cloth material and configured to be positioned on and cover the person's foot. The sleeve 12 has a general sock configuration and includes a bottom wall 14 and a perimeter wall 16 that is attached to and extends upwardly from the bottom wall 14. The perimeter wall 16 includes a heel end 18, a toe end 20, a first lateral edge 22 and a second lateral edge 24. A top wall 26 is attached to the perimeter wall 16 and has a foot opening 28 therein positioned adjacent to the heel end 18. The cloth material is air and fluid permeable. The cloth material may include conventional materials used for socks including cotton, synthetic materials, wool and the like. The cloth material may be selected depending upon the preference and manner of usage of the user and will typically be resiliently stretchable.

A coating 30 is positioned on an interior surface 32 of the sleeve 12. The coating 30 is flexible to allow the sleeve 12 to move properly when worn as a sock. The coating is air and fluid impermeable. The top wall 26 is free of the coating 30 to allow air to move through the sleeve 12 to prevent overheating, and subsequent sweating, of the foot positioned in the sleeve 12. The coating 30 may be positioned only on an upper surface 34 of the bottom wall 14 such that the perimeter wall 16 is also free of the coating 30. The coating 30 is configured to prevent the sleeve 12 from absorbing any moisturization composition positioned on a lower surface of the person's foot. The coating 30 comprises a resiliently compressible material such as, for example, a silicone gel material. Other synthetic materials including foams and gels may be utilized which are compressible but which do not flow and will not absorb oil or water-based fluids.

The coating 30 will typically at least include a heel portion 36 positioned on the bottom wall 14 and abutting the heel end 18 of the perimeter wall 16. The heel portion 36 extends between and abuts the first 22 and second 24 lateral edges. The coating 30 will further typically at least include a toe portion 38 positioned on the bottom wall 14 and abutting the toe end 20 of the perimeter wall 16. The toe portion 38 extends between and abutting the first 22 and second 24 lateral edges. As can be seen in FIG. 3, the coating 30 may fully cover the bottom wall asl well.

In use, a method of using the assembly 10 includes a person first placing a moisturizing agent on their foot. The moisturizing/medicating agent may comprise any conventional composition used to cure and or prevent dry skin as well as those compositions used for healing cracked skin. Thus, ointments, salves and lotions may be utilized. The areas of the foot most often afflicted will include the heal and toe areas. Once the agent has been placed on the foot, the foot is placed in the sleeve 12. The coating 30 will prevent the agent from being absorbed by the sleeve 12 and thereby ensure that its affects are fully directed toward the foot.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A lined foot covering assembly for retaining moisture on a person's foot, the assembly comprising:
a sleeve comprised of a cloth material and configured to be positioned on and cover the person's foot, the sleeve including a bottom wall and a perimeter wall being attached to and extending upwardly from the bottom wall, the perimeter wall including a heel end, a toe end, a first lateral edge and a second lateral edge, a top wall being attached to the perimeter wall, the top wall having a foot opening therein positioned adjacent to the heel end, the cloth material being air and fluid permeable; and
a coating being positioned on an interior surface of the sleeve, the coating being flexible, the coating being air and fluid impermeable, the top wall being free of the coating, the coating being configured to prevent the sleeve from absorbing moisturization composition positioned on a lower surface of the person's foot.

2. The lined foot covering assembly according to claim 1, wherein the coating is positioned only on an upper surface of the bottom wall.

3. The lined foot covering assembly according to claim 1, wherein the coating at least includes a heel portion being positioned on the bottom wall and abutting the heel end of the perimeter wall, the heel portion extending between and abutting the first and second lateral edges.

4. The lined foot covering assembly according to claim 3, wherein the coating includes a toe portion being positioned on the bottom wall and abutting the toe end of the perimeter wall, the toe portion extending between and abutting the first and second lateral edges.

5. The lined foot covering assembly according to claim 1, wherein the coating includes a toe portion being positioned on the bottom wall and abutting the toe end of the perimeter wall, the toe portion extending between and abutting the first and second lateral edges.

6. The lined foot covering assembly according to claim 1, wherein the coating comprised a resiliently compressible material.

7. A lined foot covering assembly for retaining moisture on a person's foot, the assembly comprising:
a sleeve comprised of a cloth material and configured to be positioned on and cover the person's foot, the sleeve including a bottom wall and a perimeter wall being attached to and extending upwardly from the bottom wall, the perimeter wall including a heel end, a toe end, a first lateral edge and a second lateral edge, a top wall being attached to the perimeter wall, the top wall having a foot opening therein positioned adjacent to the heel end, the cloth material being air and fluid permeable;
a coating being positioned on an interior surface of the sleeve, the coating being flexible, the coating being air and fluid impermeable, the top wall being free of the coating, the coating being positioned only on an upper surface of the bottom wall, the coating being configured to prevent the sleeve from absorbing moisturization composition positioned on a lower surface of the person's foot, the coating comprising a resiliently compressible material;
the coating at least including a heel portion being positioned on the bottom wall and abutting the heel end of the perimeter wall, the heel portion extending between and abutting the first and second lateral edges; and
the coating including a toe portion being positioned on the bottom wall and abutting the toe end of the perimeter wall, the toe portion extending between and abutting the first and second lateral edges.
